(19) United States
(12) Patent Application Publication
(10) Pub. No.: US 2009/0156696 A1
(43) Pub. Date: Jun. 18, 2009

(54) PROCESS FOR THE CAPTURE OF CO2 FROM CH4 FEEDSTOCK AND GTL PROCESS STREAMS

(75) Inventor: Dennis J. O'Rear, Petaluma, CA (US)

Correspondence Address: CHEVRON CORPORATION, P.O. BOX 6006, SAN RAMON, CA, 94583-0806, US

(73) Assignee: Chevron U.S.A. Inc.

(21) Appl. No.: 12/239,231

(22) Filed: Sep. 26, 2008

Related U.S. Application Data
(60) Provisional application No. 61/014,593, filed on Dec. 18, 2007.

(51) Int. Cl. C07C 1/04

(52) U.S. Cl. 518/702

(57) ABSTRACT

An integrated process is described in which at least a portion of CO₂ is captured from both the feedstock and a process stream in a GTL facility. This integrated process provides for lower costs in CO₂ capture than can be done by separate processes. At least a portion of the captured CO₂ can then be prevented from being emitted into the environment by sequestration or reaction of the CO₂ to other than a CO₂ product.

### BACKGROUND OF THE INVENTION

Carbon dioxide is a well known Greenhouse Gas, and attempts to reduce the emissions of this gas into the atmosphere are desirable. A Gas To Liquids (GTL) converts about roughly ⅔^{rd} of the starting gas (methane or natural gas) into hydrocarbonaceous liquid products with the other ⅓^{rd} being emitted as CO₂. The current costs to capture and sequester this CO₂ using conventional amine scrubbing technology coupled with sequestration of high pressure CO₂ have been estimated to be about $27/ton as shown in the CO₂ Abatement in Gas-To-Liquids Plant: Fischer-Tropsch Synthesis, IEA Greenhouse Gas R&D Program, Report Number PH3/15 November 2000. The cost of $27/ton is roughly equivalent to about $4/bbl of hydrocarbonaceous liquid product. It is desirable to reduce these costs, and several approaches have been identified as outlined in “Concepts for Reduction in CO₂ Emissions in GTL Facilities” by Dennis J. O'Rear and Fred Goede presented at the 229^{th} ACS National Meeting, Mar. 13-17, 2005 in San Diego. The source of the CO₂ in this paper are primarily the syngas streams in the GTL facility. In some situations, CO₂ is also present in the gas feedstock to a GTL facility, and capturing this CO₂ can also be important. However, processes that integrate these two operations have not been described.

### SUMMARY OF THE INVENTION

I have discovered an integrated process by which CO₂ is captured from both a GTL feedstock and the GTL process streams. This integrated process lowers the total cost for CO₂ capture in comparison to what can be achieved by processing the streams separately.

CO₂ is captured from both the gas feedstock stream and the GTL process stream by absorption. Preferably the absorption from the gas feedstock stream uses an amine scrubber at elevated pressures because this scrubbing liquid is selective for removal of both CO₂ and sulfur compounds. It is essential to remove the sulfur compounds to protect the FT catalysts used in downstream processes (syngas generation and syngas conversion). The amine scrubbing liquid is freed of CO₂ by dropping the pressure to approximately atmospheric and optionally heating the liquid to release additional CO₂. The CO₂ from the GTL process stream is also captured by absorption. Preferably the absorption from the syngas stream uses an amine-free liquid consisting of water, preferably with added basic alkali salts (preferably hydroxides). The preferred alkali salts are sodium hydroxide and or potassium hydroxide. The CO₂ from the alkali salt is freed by either reducing the pressure, increasing the temperature or both. The CO₂ from the alkali solution is combined with the CO₂ from the CO₂ from the amine system, compressed and sequestered either in an underground formation (aquifer, depleted oil or gas field, coal seam, empty salt cavern, etc). Optionally, in regions having petroleum reservoirs, the CO₂ can be used in combination with enhanced oil recovery chemicals as an enhanced oil recovery drive medium.

The process stream in the GTL facility from which CO₂ is absorbed can also include the product from steam reforming of methane or light hydrocarbons used to produce hydrogen. This hydrogen is used to upgrade the Fischer Tropsch products into fuels and/or lubricant base oils. At the exit of the steam reformer, a synthesis gas is formed that contains CO₂. The CO₂ can be extracted and used according to this invention. Preferably a non-amine scrubbing solution is used containing water and most preferably alkali metal salts, such as NaOH, KOH and combinations.

Preferably the CO₂ from water absorption system used on the syngas stream is discharged at pressures greater than atmospheric pressure. This can be done by increasing the pressure. Note, it is difficult to discharge CO₂ from amine scrubbing systems by increasing the temperature because the amine can decompose. Thus the preferred absorbent for the GTL syngas streams does not contain amines.

Compression of the CO₂ for sequestration can represent a significant cost, and steps to minimize this compression are desirable. The CO₂ from the amine absorber used on the gas feedstock can be compressed in stages. The pressure of an intermediate stage can be selected to roughly match the pressure of the CO₂ discharge from the water absorber on the GTL syngas process stream. The pressurized CO₂ from the water absorber on the GTL process stream can be combined with the intermediate pressure stream of the CO₂ from the amine scrubber. The combined streams can then be further compressed prior to sequestration. By this scheme the compression of the CO₂ from the GTL process is minimized or avoided.

### BRIEF DESCRIPTION OF THE DRAWING

FIG. 1 is an illustrative figure of a preferred CO₂ scrubbing process embodiment.

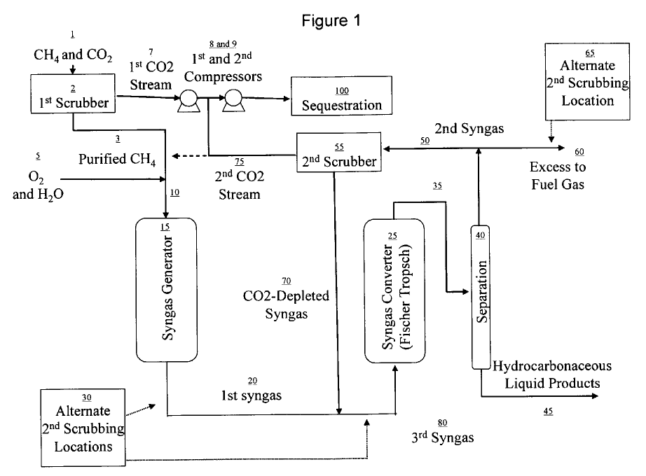

### PREFERRED EMBODIMENT

A gas stream comprising CO₂ and methane (1) is obtained. The CO₂ is removed from the gas stream by use of a first absorber that uses amine absorbent (2) to generate a purified methane stream (3) and a first CO₂-enriched stream (7). The purified methane (10) is mixed with oxygen and optionally steam (5) and reacted in a synthesis gas formation reactor (15) to form a first synthesis gas (20). This is mixed with a CO₂-depleted synthesis gas stream (70) to form a third synthesis gas stream (80). The third synthesis gas stream is reacted over a synthesis gas conversion reactor, preferably a Fischer Tropsch reactor (25) to form one or more effluent streams (35). The effluent or effluent streams (35) are separated in a separator (40) to form a hydrocarbonaceous product (45) and a second synthesis gas stream (50). At least a portion of the second synthesis gas is purified in a second absorber (55) to produce the CO₂-depleted synthesis gas stream and a 2^{nd} CO₂-enriched stream at pressure P where P is greater than atmospheric but less than or equal to the pressure of the 2^{nd} synthesis gas stream. Optionally excess of the second synthesis gas can be used as fuel gas (60). The first CO₂-enriched steam is compressed in a first compressor (8) and then a second compressor (9) where the inlet pressure to the 2^{nd} compressor is less than or equal to P. The second CO₂-enriched stream is mixed with the first gas stream ahead of the inlet of the 2^{nd} compressor preferably in between the first and send compressors.

At least some or all captured CO₂ is sequestered. Alternatively, some or all of the CO₂ may be further reacted to a product other than CO2 for use or storage.

While the invention was described with respect to preferred embodiments, modifications apparent to the ordinary skilled artisan are contemplated to be within the scope of the invention.

1. A process to convert a gas stream containing both CO₂ and methane into hydrocarbonaceous liquids in a GTL facility with CO₂ capture comprising:
a. obtaining a gas stream containing both CO₂ and methane;
b. absorbing at least a portion of the CO₂ from the methane by use of an amine absorbent;
c. discharging at least a portion of the CO₂ from the amine absorbent to obtain a first CO₂ enriched stream;
d. converting at least a portion of the methane into a first synthesis gas and CO₂;
e. reacting the first synthesis gas over a synthesis gas conversion process to form a hydrocarbonaceous liquid and a second synthesis gas;
f. absorbing at least a portion of the CO₂ from a synthesis gas stream of pressure P by use of a second absorbent wherein the synthesis gas stream is selected from the group consisting of the first synthesis gas stream, the second synthesis gas stream, and combinations;
g. discharging at least a portion of the CO₂ from the second absorbent to obtain a second CO₂ enriched stream;
h. combining at least a portion of the first and second CO₂ enriched streams; and
i. sequestering at least a portion of the combined streams.

2. A process according to claim 1 wherein the second absorbent comprises water and essentially no amine.

3. A process according to claim 2 wherein the second absorbent contains alkali metals.

4. A process according to claim 2 where the discharge pressure of the second absorbent is at pressures greater than atmospheric.

5. A process according to claim 4 wherein the discharge pressure of the second absorbent is greater than atmospheric and less than or equal to P.

6. A process according to claim 1 further comprising the use of staged compression with at least one intermediate pressure.

7. A process according to claim 6 wherein the intermediate pressure is equal to or less than the discharge pressure of the second absorbent.

8. A process according to claim 1 wherein a third synthesis gas stream is formed by blending at least a portion of the first synthesis gas and at least a portion of the second synthesis gas, and wherein the third synthesis gas is reacted in the synthesis gas conversion facility.

9. A process according to claim 8 wherein the CO₂ is removed from the third synthesis gas stream by use of the second absorbent.

10. A process according to claim 1 further comprising the manufacture of hydrogen from methane by steam reforming and from which CO₂ is recovered and combined with the first and second streams.
